(19) United States
(12) Patent Application Publication
(10) Pub. No.: US 2019/0216966 A1
(43) Pub. Date: Jul. 18, 2019

(54) TOILET DEODORANT COMPOSITION AND METHOD

(71) Applicants: Jonathan Andrew Mancini, Maple Ridge (CA); Ryan Gorin Lalonde, Vancouver (CA)

(72) Inventors: Jonathan Andrew Mancini, Maple Ridge (CA); Ryan Gorin Lalonde, Vancouver (CA)

(21) Appl. No.: 15/873,494

(22) Filed: Jan. 17, 2018

(51) Int. Cl. A61L 9/012; C11D 7/26; C11D 7/12; C11D 7/44; C11D 7/50

(52) U.S. Cl. CPC A61L 9/012; C11D 7/265; C11D 7/5022; C11D 7/268; C11D 7/44; C11D 7/12

(57) ABSTRACT

A chemical composition includes citric acid, sodium bicarbonate, starch, at least one oil, and a liquid composition such as water combined to form a solid tablet. The composition may react with water to produce a film over a body of water, such that the film is used to cover the surface of the contents of a latrine and prevent gasses and odors from escaping the latrine.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of cleaning compositions of existing art and more specifically relates to deodorants.

### RELATED ART

A latrine is a generic term for a toilet or other facility used as a toilet. Latrines provide significant sanitary advantages as a site for defecation, and help prevent the propagation of disease. Unfortunately, latrines can also concentrate odors produced by defecation, and provide an unpleasant environment in general to a user. While modern flushing toilets may minimize the presence of odors, some odors will inevitably remain in the area around the latrine. A suitable solution is desired.

U.S. Pat. No. 3,762,875 to H. Burmeister relates to an odor sealing method. The described odor sealing method includes an odor-sealing composition which contains a foam-forming agent which forms an odor-sealing foam cushion on contact with water. The composition may additionally contain deodorizing, odor-improving, disinfecting, and/or cleaning additives. It is used in liquid, pulverulent, granulated, or compressed shaped form or as an aerosol.

### SUMMARY OF THE INVENTION

In view of the foregoing disadvantages inherent in the known deodorant art, the present disclosure provides a novel toilet deodorant composition and method. The general purpose of the present disclosure, which will be described subsequently in greater detail, is to provide an efficient and effective toilet deodorant composition and method.

A chemical composition and method is disclosed herein. The chemical composition includes citric acid, sodium bicarbonate, starch, at least one oil, and a liquid composition such as water combined to form a (preferable) solid tablet. The composition may react with water to produce a film over a body of water, such that the film is used to cover the surface of the contents of a latrine and prevent gasses and odors from escaping the latrine.

According to another embodiment, a method of controlling odors in a latrine is also disclosed herein. The method of controlling odors in a latrine includes firstly, providing the chemical composition; secondly, depositing the solid aggregate within a latrine containing water; thirdly, dissolving the chemical composition within the water; fourthly, forming a film over the surface of the water to conceal the contents of the latrine; and fifthly, containing gases within the latrine underneath the film.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a toilet deodorant composition and method, constructed and operative according to the teachings of the present disclosure.

FIG. 1 is a perspective view of the chemical composition during an ‘in-use’ condition, according to an embodiment of the disclosure.

FIG. 2 is a perspective view of the chemical composition of FIG. 1, according to an embodiment of the present disclosure.

FIG. 3 is a perspective view of the chemical composition of FIG. 1, according to an embodiment of the present disclosure.

FIG. 4 is a statistical graphic of the chemical composition of FIG. 1, according to an embodiment of the present disclosure.

FIG. 5 is a flow diagram illustrating a method of use for deodorizing a toilet, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a deodorant means and more particularly to a toilet deodorant composition and method as used to improve the deodorizing a toilet.

Generally, the toilet deodorant composition includes a combination of ingredients configured to react in water to produce a film product for covering the contents of a toilet. The composition may be formed into a tablet which can be easily deposited in a toilet. When the tablet enters the toilet, the composition reacts and forms a cohesive film which covers the surface of the water and any waste contained in the toilet. The film may conceal any unsightly waste and may also contain gasses within the toilet to control odors.

The deodorant composition, in a preferred embodiment, may constitute the combination of one-half cup of sodium bicarbonate, or baking soda; one-quarter cup of citric acid; one-quarter cup of starch; one tablespoon of an essential oil; one tablespoon of an additional oil; one tablespoon of water; and a few drops of a coloring agent; or any combination of these ingredients with proportional portions. Other compositions are envisioned and included within the scope of this disclosure. Preferably, the tablet is formed as a cylinder (3D or other suitable shape) having a small enough form factor to easily fit within a user's hand. The tablets may be manufactured using a molding process.

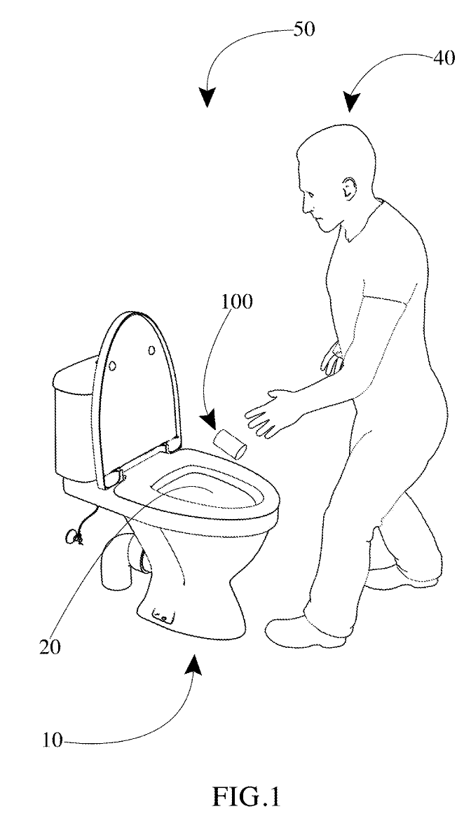

Referring now more specifically to the drawings by numerals of reference, there is shown in FIGS. 1-4, various views of a chemical composition 100.

FIG. 1 shows a chemical composition 100 during an ‘in-use’ condition 50, according to an embodiment of the present disclosure. Here, chemical composition 100 may be beneficial for use by user 40 40 to deodorize a toilet. As illustrated, chemical composition 100 may be configured to be used by user 40 to deodorize latrine 10. Chemical composition 100 may be thrown by user 40 into body of water 20 within latrine 10, chemical composition 100 being configured to react with body of water 20 upon contact. Upon reading this specification, it should be appreciated that, under appropriate circumstances, considering such issues as user preferences, design preference, structural requirements, marketing preferences, cost, available materials, technological advances, etc., other structural arrangements such as, for example, tablet shapes, encapsulating structures additional chemical additives, etc., may be sufficient.

According to one embodiment, the chemical composition 100 may be arranged as a kit 105. In particular, the chemical composition 100 may further include a set of instructions 107. The instructions 107 may detail functional relationships in relation to the structure of the chemical composition 100 such that the chemical composition 100 can be used, maintained, or the like, in a preferred manner.

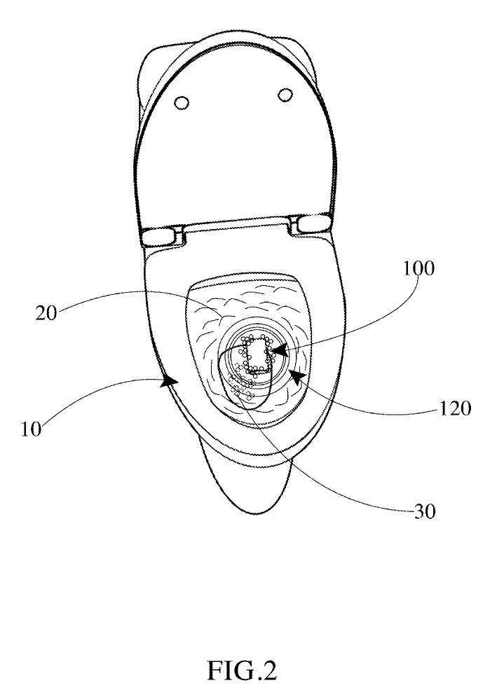

FIG. 2 shows chemical composition 100 of FIG. 1, according to an embodiment of the present disclosure. As above, chemical composition 100 may be configured to be thrown/placed into latrine 10. Chemical composition 100 is configured to react with a body of water 20 to produce cohesive film 120 over body of water 20 within latrine 10 configured to prevent gases 30 from escaping body of water 20.

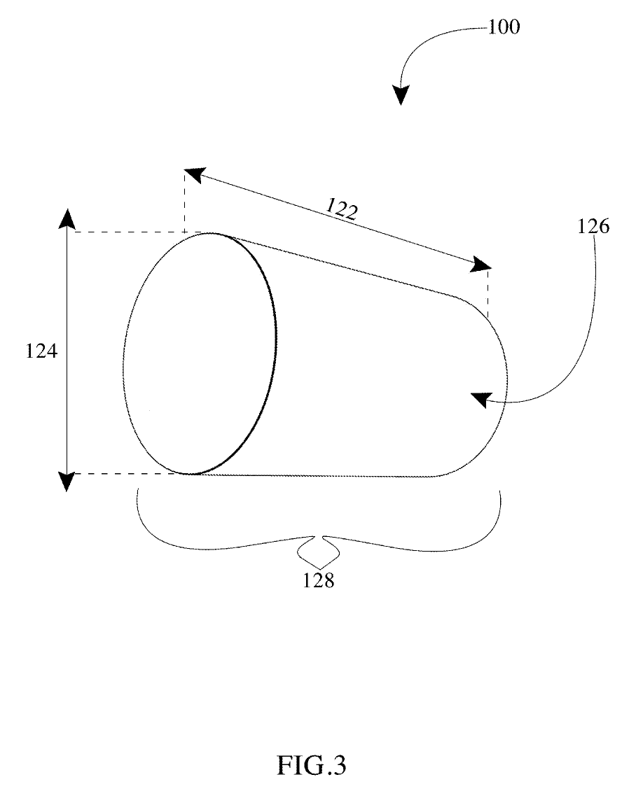

FIG. 3 is a perspective view of the chemical composition 100 of FIG. 1, according to an embodiment of the present disclosure. Chemical composition 100 may be formed as a solid aggregate 128 (or non-solid), being molded into a substantially cylindrical tablet having a cylinder-length 122 and a cylinder-diameter 124. Chemical composition 100 further includes color additive 126 configured to alter a hue of solid aggregate 128. Cylinder-length 122 may be less than one inch, and cylinder-diameter 124 may be less than one inch, solid aggregate 128 being configured to be easily carried by user 40 (FIG. 1),

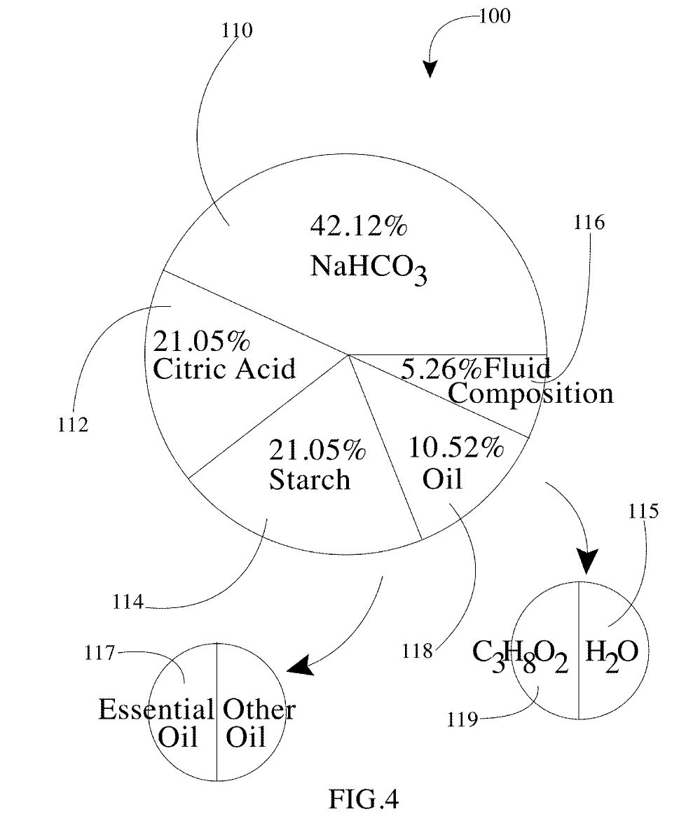

Referring now to FIG. 4, a chart of the chemical composition 100 of FIG. 1, according to an embodiment of the present disclosure. Chemical composition 100 may include sodium bicarbonate 110, citric acid 112, starch 114, at least one oil 116, and liquid composition 118. Liquid composition 118 (or non-liquid) may include at least one of dihydrogen oxide 115 and propylene glycol 119. Preferably, at least one oil 116 includes at least one essential oil 117. At least one oil 116 may include about fifty-percent of at least one essential oil 117. In addition, at least one oil 116 may include one or more of olive oil and an aroma oil. Starch 114 may include a corn-derived starch, a wheat-derived starch, a rice-derived starch, or a tapioca-derived starch for example. In particular, chemical composition 100 may, in preferred embodiments, be composed of 21.05 percent citric acid 112, 42.12 percent sodium bicarbonate 110, 21.05 percent starch 114, 10.52 percent at least one oil 118, and 5.26 percent of the fluid composition 116.

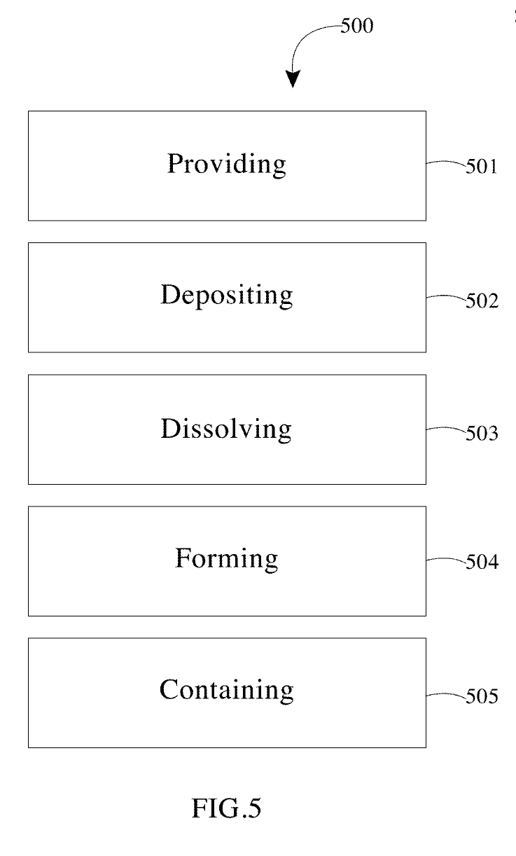

FIG. 5 is a flow diagram illustrating a method for deodorizing a toilet 500, according to an embodiment of the present disclosure. In particular, the method for deodorizing a toilet 500 may include one or more components or features of the chemical composition 100 as described above. As illustrated, the method for deodorizing a toilet 500 may include the steps of: step one 501, providing a chemical composition configured to deodorize a space, the chemical composition comprising citric acid, sodium bicarbonate, a starch, at least one oil, and polypropylene glycol, wherein the chemical composition reacts with water to produce a cohesive film over a body of water configured to prevent gases from escaping the body of water, and wherein the chemical composition comprises a solid aggregate having the shape of a substantially cylindrical tablet; step two 502, depositing the solid aggregate within a latrine containing water; step three 503, dissolving the chemical composition within the water; step four 504, forming a cohesive film over the surface of the water; and step five 505, containing gases within the latrine underneath the cohesive film.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for deodorizing a toilet, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A chemical composition configured to deodorize a space, the chemical composition comprising:
citric acid;
sodium bicarbonate;
a starch;
at least one oil; and
a liquid composition;
wherein the chemical composition reacts with water to produce a cohesive film over a body of water configured to prevent gases from escaping the body of water.

2. The chemical composition of claim 1, wherein the liquid composition includes at least one of dihydrogen oxide and propylene glycol.

3. The chemical composition of claim 1, wherein the at least one oil includes olive oil.

4. The chemical composition of claim 1, wherein the at least one oil includes an aroma oil.

5. The chemical composition of claim 1, wherein the at least one oil includes at least one essential oil.

6. The chemical composition of claim 1, wherein the starch is a corn-derived starch.

7. The chemical composition of claim 1, wherein the starch is a wheat-derived starch.

8. The chemical composition of claim 1, wherein the starch is a rice-derived starch.

9. The chemical composition of claim 1, wherein the starch is a tapioca-derived starch.

10. The chemical composition of claim 1, wherein the chemical composition further includes a fragrance agent configured to mask odors.

11. The chemical composition of claim 1, wherein the chemical composition comprises a solid aggregate having the shape of a substantially cylindrical tablet having a cylinder-length and a cylinder-diameter.

12. The chemical composition of claim 11, wherein the chemical composition further includes a color additive configured to alter the hue of the solid aggregate.

13. The chemical composition of claim 11, wherein the cylinder-length is less than one inch, the solid aggregate being configured to be easily carried by a user.

14. The chemical composition of claim 11, wherein the cylinder-diameter is less than one inch, the solid aggregate being configured to be easily carried by a user.

15. The chemical composition of claim 2, wherein the chemical composition further comprises:
21.05 percent citric acid;
42.12 percent sodium bicarbonate;
21.05 percent starch;
10.52 percent oil; and
5.26 percent of the fluid composition.

16. The chemical composition of claim 15, wherein the at least one oil includes fifty-percent of at least one essential oil.

17. A toilet-deodorizing chemical composition, the chemical composition comprising:
citric acid;
sodium bicarbonate;
a starch;
at least one oil; and
a liquid composition;
wherein the chemical composition reacts with water to produce a cohesive film over a body of water configured to prevent gases from escaping the body of water;
wherein the liquid composition includes at least one of dihydrogen oxide and propylene glycol;
wherein the at least one oil includes at least one essential oil.
wherein the starch is a corn-derived starch;
wherein the chemical composition comprises a solid aggregate having the shape of a substantially cylindrical tablet having a cylinder-length and a cylinder-diameter;
wherein the chemical composition further includes a color additive configured to alter the hue of the solid aggregate;
wherein the cylinder-length is less than one inch, the solid aggregate being configured to be easily carried by a user;
wherein the cylinder-diameter is less than one inch, the solid aggregate being configured to be easily carried by a user;
wherein the chemical composition further comprises:
about 21.05 percent citric acid;
about 42.12 percent sodium bicarbonate;
about 21.05 percent starch;
about 10.52 percent oil; and
about 5.26 percent of the fluid composition; and
wherein the at least one oil includes fifty-percent of at least one essential oil.

18. The chemical composition of claim 17, further comprising set of instructions; and
wherein the chemical composition is arranged as a kit.

19. A method of controlling odors within a latrine, the method comprising the steps of:
providing a chemical composition configured to deodorize a space, the chemical composition comprising:
citric acid;
sodium bicarbonate;
a starch;
at least one oil; and
polypropylene glycol;
wherein the chemical composition reacts with water to produce a cohesive film over a body of water configured to prevent gases from escaping the body of water; and
wherein the chemical composition comprises a solid aggregate having the shape of a substantially cylindrical tablet;
depositing the solid aggregate within a latrine containing water;
dissolving the chemical composition within the water;
forming a cohesive film over the surface of the water; and
containing gases within the latrine underneath the cohesive film.
